(19) United States
(12) Patent Application Publication
(10) Pub. No.: US 2014/0350023 A1
(43) Pub. Date: Nov. 27, 2014

(54) AMORPHOUS FORM OF SITAGLIPTIN SALTS

(71) Applicant: RANBAXY LABORATORIES LIMITED, New Delhi, Delhi (IN)

(72) Inventors: Suresh Babu Jayachandra, Gurgaon (IN); Jigar Bhaskarbhai Shah, Bharuch (IN); Chiguru Sailu, Karimnagar (IN); Sandeep Thakaji Kanawade, Ahmednagar (IN)

(21) Appl. No.: 14/363,959

(22) PCT Filed: Dec. 7, 2012
(86) PCT No.: PCT/IB2012/057086

§ 371 Date: Jun. 9, 2014

(30) Foreign Application Priority Data
Dec. 8, 2011 (IN) 3577/DEL/2011

(51) Int. Cl. C07D 487/04

(52) U.S. Cl. CPC C07D 487/04
USPC 514/249; 544/350

(57) ABSTRACT

The present invention claims amorphous solid state forms of sitagliptin salts, processes for their preparation, and pharmaceutical compositions thereof. As salt forming anions are claimed: The maleate, fumarate, besylate, mesylate and succinate.

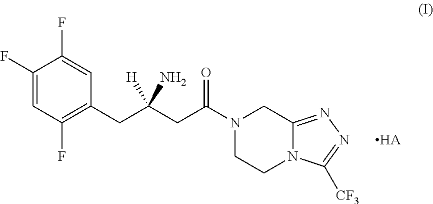

### FIELD OF THE INVENTION

The present invention provides amorphous forms of sitagliptin salts, processes for their preparation, and pharmaceutical compositions thereof.

### BACKGROUND OF THE INVENTION

Sitagliptin dihydrogen phosphate monohydrate of Formula A, an orally-active inhibitor of the dipeptidyl peptidase-4 (DPP-4) enzyme, chemically designated as 7-[(3R)-3-amino- 1-oxo-4-(2,4,5-trifluorophenyl)butyl] -5,6,7,8-tetrahydro-3 -(trifluoromethyl)-1,2,4-triazolo[4,3-a]pyrazine phosphate (1:1) monohydrate, is indicated as an adjunct therapy to diet and exercise to improve glycemic control in adults with type 2 diabetes mellitus.

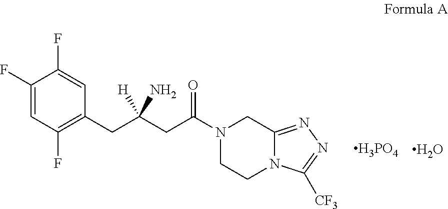

U.S. Pat. No. 6,699,871 (hereinafter “the '871 patent”), in particular Example 7, provides a process for the preparation of a sitagliptin hydrochloride salt. A list of pharmaceutically acceptable salts is generally included in the '871 patent.

U.S. Pat. No. 7,326,708 provides a process for the preparation of crystalline sitagliptin dihydrogenphosphate monohydrate.

PCT Publication No. WO 2005/072530 provides a process for the preparation of crystalline salts of sitagliptin with hydrochloric acid, benzene sulfonic acid, p-toluene sulfonic acid, D- and L-tartaric acid and (1S)-(+)- and (1R)-(-)-10-camphorsulfonic acid.

PCT Publication No. WO 2005/030127 provides a process for the preparation of sitagliptin dihydrogenphosphate anhydrate Form IV. It also provides a process for the preparation of sitagliptin dihydrogen phosphate anhydrate Form I by heating sitagliptin dihydrogenphosphate anhydrate Form IV at a temperature above 140° C. for about 1 hour.

PCT Publication No. WO 2005/020920 provides a process for the preparation of crystalline anhydrate Form I, crystalline desolvated anhydrate Form II, crystalline anhydrate Form III, and a crystalline ethanol solvate of sitagliptin dihydrogen phosphate. It also provides a process for the preparation of a mixture of sitagliptin dihydrogen phosphate anhydrate Form I and anhydrate Form III.

PCT Publication No. WO 2007/035198 provides a process for the preparation of a dodecylsulfate salt of sitagliptin.

PCT Publication No. WO 2008/000418 provides a process for the preparation of sitagliptin hydrochloride in amorphous form.

PCT Publication No. WO 2009/120746 provides processes for the preparation of a crystalline form of sitagliptin phosphate, characterized by a powder XRD pattern with peaks at about 4.7, 13.5, 17.7, 18.3, and 23.7 ±0.2° 20.

PCT Publication No. WO 2006/033848 provides a process for the preparation of crystalline sitagliptin dihydrogenphosphate monohydrate and amorphous sitagliptin dihydrogenphosphate.

U.S. Publication No. U.S. 2009/247532 provides processes for the preparation of polymorph Form V of crystalline sitagliptin phosphate and polymorph Form I of sitagliptin phosphate.

PCT Publication No. WO 2009/084024 provides a process for the preparation of R-sitagliptin dibenzyl-L-tartrate.

PCT Publication No. WO 2009/085990 provides a process for the preparation of crystalline anhydrate Form A of sitagliptin dihydrogen phosphate, crystalline sitagliptin sulfate, crystalline sitagliptin hydrobromide, crystalline sitagliptin methane sulfonate, crystalline sitagliptin acetate, crystalline sitagliptin benzoate, crystalline sitagliptin oxalate, crystalline sitagliptin succinate, crystalline sitagliptin mandelate, crystalline sitagliptin fumarate and crystalline sitagliptin lactate.

PCT Publication No. WO 2010/032264 provides a process for the preparation of crystalline Form 3 of sitagliptin, a crystalline form of dibenzoyl-L-tartaric acid salt of sitagliptin, an amorphous form of sitagliptin and a crystalline form of sitagliptin phosphate.

PCT Publication No. WO 2010/000469 provides a process for the preparation of sitagliptin hydrochloride Form L sitagliptin hydrochloride Form II, sitagliptin fumarate Form I, sitagliptin fumarate form II, sitagliptin malate, sitagliptin sulfate Form 1, sitagliptin sulfate Form II, sitagliptin phosphate, sitagliptin succinate Form I, sitagliptin succinate Form II, sitagliptin succinate Form III, sitagliptin lactate, sitagliptin glycolate, sitagliptin maleate Form I, sitagliptin maleate Form II, crystalline sitagliptin citrate, amorphous sitagliptin citrate, sitagliptin mesylate Form I and sitagliptin mesylate Form II.

PCT Publication No. WO 2010/012781 provides a process for the preparation of sitagliptin galactarate, sitagliptin hemi-L-malate, sitagliptin D-gluconate, sitagliptin succinate, sitagliptin hydrobromide, sitagliptin thiocyanate, sitagliptin oxalate, sitagliptin aspartate, sitagliptin ethanedisulfonate, sitagliptin pyroglutamate, sitagliptin glutarate, sitagliptin acetate, sitagliptin hydrochloride amorphous form, sitagliptin citrate amorphous form, sitagliptin hemicitrate amorphous form, sitagliptin glycolate amorphous form and sitagliptin maleate amorphous form.

PCT Publication No. WO 2010/117738 provides a process for the preparation of crystalline Form S1 of sitagliptin sulfate, crystalline Form S2 of sitagliptin sulfate, crystalline Form S3 of sitagliptin sulfate, crystalline Form S4 of sitagliptin sulfate, crystalline Form S5 of sitagliptin sulfate, crystalline Form S6 of sitagliptin sulfate, crystalline Form S7 of sitagliptin sulfate, crystalline Form S8 of sitagliptin sulfate, crystalline Form D1 of sitagliptin (+)-dibenzoyl-tartrate, crystalline Form D2 of sitagliptin (+)-dibenzoyl-tartrate, crystalline Form F1 of sitagliptin fumarate, crystalline Form F2 of sitagliptin fumarate, crystalline Form Ml of sitagliptin (D)-(+)-malate, crystalline Form M2 of sitagliptin (D)-(+)-malate, crystalline Form 11 of sitagliptin L-malate, crystalline Form O1 of sitagliptin oxalate, crystalline Form O2 of sitagliptin oxalate, crystalline Form Q1 of sitagliptin quinate, crystalline Form U1 of sitagliptin succinate, crystalline Form E1 of sitagliptin acetate, crystalline Form A1 of sitagliptin maleate, crystalline Form N1 of sitagliptin (S)-mandelate, crystalline Form N2 of sitagliptin (S)-mandelate, crystalline Form N3 of sitagliptin (S)-mandelate, crystalline Form N4 of sitagliptin (S)-mandelate, amorphous sitagliptin mandelate, crystalline Form N5 of sitagliptin (R)-mandelate, crystalline Form N6 of sitagliptin (R)-mandelate, crystalline Form L1 of sitagliptin lactate, crystalline Form L2 of sitagliptin lactate, crystalline Form L3 of sitagliptin lactate, crystalline Form L4 of sitagliptin lactate, and amorphous sitagliptin orotate.

PCT Publication No. WO 2010/092090 provides a process for the preparation of crystalline sitagliptin D-glucuronate, crystalline sitagliptin glutarate, crystalline sitagliptin hydrogen sulfate, crystalline sitagliptin L-lactate, crystalline sitagliptin oxalate, sitagliptin caprate, sitagliptin L-mandelate, and sitagliptin ethanesulfonate.

PCT Publication No. WO 2010/122578 provides a process for the preparation of sitagliptin hydrogen phosphate monohydrate and sitagliptin mandalate.

PCT Publication No. WO 2011/025932 provides a process for the preparation of sitagliptin phosphate and sitagliptin hydrochloride.

PCT Publication No. WO 2011/060213 provides a process for the preparation of sitagliptin phosphate and sitagliptin formate.

PCT Publication No. WO 2011/018494 provides a process for the preparation of sitagliptin fumarate.

Journal of Medicinal Chemistry, 48(1), 141-151 (2005), provides a process for the preparation of sitagliptin hydrochloride and sitagliptin fumarate.

Several processes are known in the literature for making sitagliptin or a salt thereof, for example, PCT Publication Nos. WO 2011/049344, WO 2010/131025, WO 2010/078440, WO 2004/083212, WO 2006/065826, WO 2010/097420, WO 2004/080958, WO 2004/087650 and WO 2004/085661.

In the pharmaceutical industry, there is a constant need to identify the critical physicochemical parameters such as novel salts and novel polymorphic forms that affect the drug's performance, stability, etc., which may play a key role in determining a drug's market acceptance and success.

The term “polymorphism” includes different physical forms, crystal forms, and crystalline/liquid crystalline/non-crystalline (amorphous) forms. It has been observed that many antibiotics, antibacterials, tranquilizers, etc., exhibit polymorphism. Some polymorphic forms of a given drug exhibit superior bioavailability, and consequently show much higher activity compared to other polymorphs. It is also known that the amorphous forms in a number of drugs exhibit different dissolution characteristics and in some cases, different bioavailability patterns as compared to the crystalline form. For some therapeutic indications, one bioavailability pattern may be favored over another.

It is therefore important to evaluate polymorphism of drug substances. Therefore there is also a strong need for developing amorphous forms of salts of sitagliptin. There is no specific disclosure in the above references about the amorphous forms of sitagliptin salts of the present invention.

### SUMMARY OF THE INVENTION

A first aspect of the present invention provides an amorphous form of a compound of Formula I

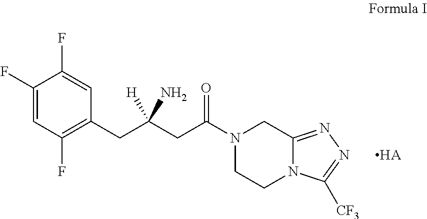

wherein HA is selected from the group consisting of maleic acid, fumaric acid, benzenesulfonic acid, methanesulfonic acid, and succinic acid.

A second aspect of the present invention provides a process for the preparation of an amorphous form of a compound of Formula I

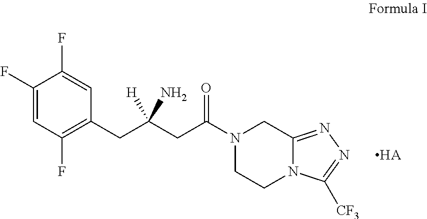

wherein HA is selected from the group consisting of maleic acid, fumaric acid, benzenesulfonic acid, methanesulfonic acid, and succinic acid, the process comprising:

a) treating sitagliptin with HA wherein HA is selected from the group consisting of maleic acid, fumaric acid, benzenesulfonic acid, methanesulfonic acid, and succinic acid; and

b) isolating an amorphous form of a compound of Formula I.

A third aspect of the present invention provides an amorphous form of a compound of Formula I

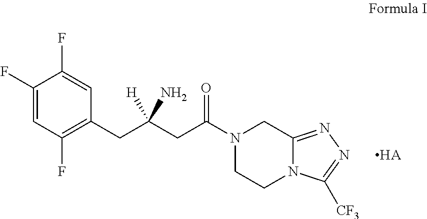

wherein HA is selected from the group consisting of maleic acid, fumaric acid, benzenesulfonic acid, methanesulfonic acid, and succinic acid for the preparation of the sitagliptin base, other salts, solvates or polymorphs thereof.

A fourth aspect of the present invention provides a pharmaceutical composition comprising an amorphous form of the compound of Formula I

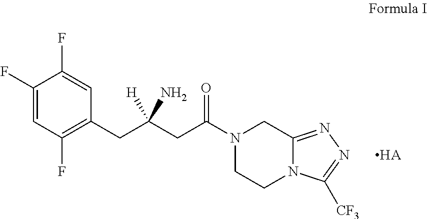

wherein HA is selected from the group consisting of maleic acid, fumaric acid, benzenesulfonic acid, methanesulfonic acid, and succinic acid, and a pharmaceutically acceptable carrier.

A fifth aspect of the present invention provides a method of treating or preventing type 2 diabetes mellitus which comprises administering to a patient in need thereof a therapeutically effective amount of an amorphous form of the compound of Formula I

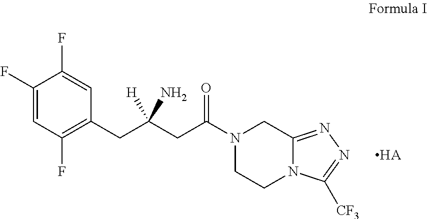

wherein HA is selected from the group consisting of maleic acid, fumaric acid, benzenesulfonic acid, methanesulfonic acid, and succinic acid, and a pharmaceutically acceptable carrier.

### DETAILED DESCRIPTION OF THE INVENTION

Sitagliptin prepared by any of the methods known in the art including those described in, for example, U.S. Pat. Nos. 6,699,871, and 7,326,708 and PCT Publication Nos. WO 2010/131025, WO 2004/083212, WO 2010/097420, WO 2004/087650, WO 2004/085661, WO 2005/072530, WO 2005/030127, WO 2005/020920, WO 2007/035198, WO 2006/033848, WO 2009/085990, WO 2009/084024, WO 2010/032264, WO 2010/117738, and WO 2010/122578 may be used as the starting material.

A first aspect of the present invention provides an amorphous form of the compound of Formula I

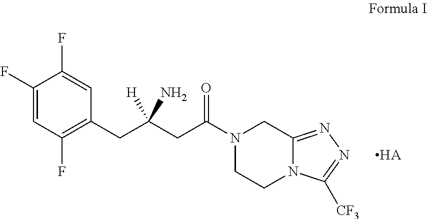

wherein HA is selected from the group consisting of maleic acid, fumaric acid, benzenesulfonic acid, methanesulfonic acid, and succinic acid.

The term “amorphous” refers to a solid without a long-range crystalline order. An amorphous form of the compound of Formula I of the present invention preferably contains less than about 20% crystalline forms, more preferably less than 5% crystalline forms, and still more preferably is essentially free of crystalline forms. “Essentially free of crystalline forms” means that no crystalline polymorph forms can be detected within the limits of an X-ray Powder Diffractometer.

An amorphous form of sitagliptin maleate of the present invention may be characterized by an XRPD pattern substantially the same as depicted in FIG. 1.

An amorphous form of sitagliptin maleate of the present invention may be characterized by FTIR as depicted in FIG. 2.

An amorphous form of sitagliptin fumarate of the present invention may be characterized by an XRPD pattern substantially the same as depicted in FIG. 3.

An amorphous form of sitagliptin fumarate of the present invention may be characterized by FTIR as depicted in FIG. 4.

An amorphous form of sitagliptin benzenesulfonate of the present invention may be characterized by an XRPD pattern substantially the same as depicted in FIG. 5.

An amorphous form of sitagliptin benzenesulfonate of the present invention may be characterized by FTIR as depicted in FIG. 6.

An amorphous form of sitagliptin methanesulfonate of the present invention may be characterized by an XRPD pattern substantially the same as depicted in FIG. 7.

An amorphous form of sitagliptin methanesulfonate of the present invention may be characterized by FTIR as depicted in FIG. 8.

An amorphous form of sitagliptin succinate of the present invention may be characterized by an XRPD pattern substantially the same as depicted in FIG. 9.

An amorphous form of sitagliptin succinate of the present invention may be characterized by FTIR as depicted in FIG. 10.

A second aspect of the present invention provides a process for the preparation of an amorphous form of the compound of Formula I

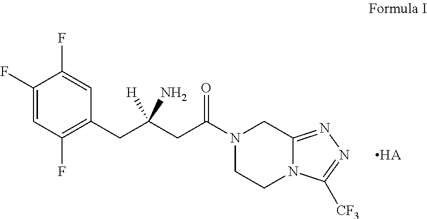

wherein HA is selected from the group consisting of maleic acid, fumaric acid, benzenesulfonic acid, methanesulfonic acid, and succinic acid, the process comprising:

a) treating sitagliptin with HA wherein HA is selected from the group consisting of maleic acid, fumaric acid, benzenesulfonic acid, methanesulfonic acid, and succinic acid; and

b) isolating an amorphous form of the compound of Formula I.

Step a) of treating sitagliptin with HA may include adding, dissolving, slurrying, stirring or a combination thereof. Sitagliptin may be treated with HA in a suitable solvent at a temperature of about 20° C. to about 80° C. for a time period sufficient to complete the reaction.

Step a) in one embodiment involves adding HA to sitagliptin in one or more solvents at a temperature of about 20° C. to about 80° C., optionally while stirring. HA may be added in one lot, or can be added in two or more portions, or can be added incrementally. After HA has been added, the resultant mixture may be stirred for about 15 minutes to about 3 hours at a temperature of about 20° C. to about 80° C.

The term “solvent” includes any solvent or solvent mixture including, for example, water, esters, alkanols, halogenated hydrocarbons, ketones, ethers, polar aprotic solvents, or mixtures thereof.

Examples of the esters may include ethyl acetate, n-propyl acetate, isopropyl acetate, and n-butyl acetate. Examples of alkanols include those primary, secondary and tertiary alcohols having from one to six carbon atoms. Examples of suitable alkanol solvents include methanol, ethanol, n-propanol, isopropanol and butanol. Examples of halogenated hydrocarbons include dichloromethane, chloroform, and 1,2-dichloroethane. Examples of ketones include acetone, methyl ethyl ketone, and the like. Examples of ethers include diethyl ether, tetrahydrofuran, and the like. Examples of a suitable polar aprotic solvent include N,N-dimethylformamide, N,N-dimethylacetamide, dimethylsulphoxide, acetonitrile and N-methylpyrrolidone.

Step b) of isolating a compound of Formula I comprises common isolation techniques such as evaporation, evaporation under vacuum, cooling, extraction, one or more of washing, crystallization, precipitation, filtration, filtration under vacuum, decantation and centrifugation, or a combination thereof.

A third aspect of the present invention provides an amorphous form of the compound of Formula I

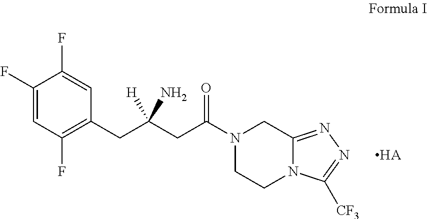

wherein HA is selected from the group consisting of maleic acid, fumaric acid, benzenesulfonic acid, methanesulfonic acid, and succinic acid for the preparation of sitagliptin base, other salts, solvates or polymorphs thereof.

The compound of Formula I may be used for preparation of sitagliptin by contacting with a base. The base may be selected from the group comprised of hydroxides, carbonates and bicarbonates of alkali and alkaline earth metals, ammonia, alkyl amines, hydrazine, and the like. Examples of hydroxides, carbonates and bicarbonates of alkali and alkaline earth metals may include lithium hydroxide, sodium hydroxide, potassium hydroxide, sodium carbonate, potassium carbonate, sodium bicarbonate or potassium bicarbonate. Examples of alkyl amines may include diethyl amine, triethyl amine or methyl diethyl amine. Sitagliptin thus obtained may be converted to other salts, solvates or polymorphs thereof.

A fourth aspect of the present invention provides a pharmaceutical composition comprising an amorphous form of the compound of Formula I

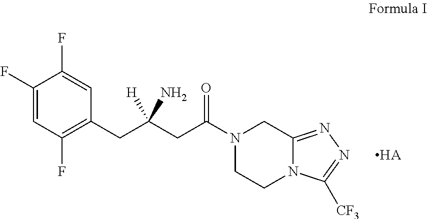

wherein HA is selected from the group consisting of maleic acid, fumaric acid, benzenesulfonic acid, methanesulfonic acid, and succinic acid, and a pharmaceutically acceptable carrier.

A fifth aspect of the present invention provides a method of treating or preventing type 2 diabetes mellitus which comprises administering to a patient in need thereof a therapeutically effective amount of an amorphous form of the compound of Formula I

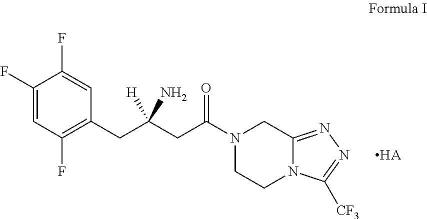

wherein HA is selected from the group consisting of maleic acid, fumaric acid, benzenesulfonic acid, methanesulfonic acid, and succinic acid, and a pharmaceutically acceptable carrier.

### BRIEF DESCRIPTION OF THE FIGURES

FIG. 1 depicts the X-Ray Powder Diffractogram (XRPD) of sitagliptin maleate prepared as per Example 1.

FIG. 2 depicts the Fourier-Transform Infra-Red (FTIR) spectrum of sitagliptin maleate prepared as per Example 1.

FIG. 3 depicts the X-Ray Powder Diffractogram (XRPD) of sitagliptin fumarate prepared as per Example 2.

FIG. 4 depicts the Fourier-Transform Infra-Red (FTIR) spectrum of sitagliptin fumarate prepared as per Example 2.

FIG. 5 depicts the X-Ray Powder Diffractogram (XRPD) of sitagliptin benzenesulfonate prepared as per Example 3.

FIG. 6 depicts the Fourier-Transform Infra-Red (FTIR) spectrum of sitagliptin benzenesulfonate prepared as per Example 3.

FIG. 7 depicts the X-Ray Powder Diffractogram (XRPD) of sitagliptin methanesulfonate prepared as per Example 4.

FIG. 8 depicts the Fourier-Transform Infra-Red (FTIR) spectrum of sitagliptin methanesulfonate prepared as per Example 4.

FIG. 9 and FIG. 9a depict the X-Ray Powder Diffractogram (XRPD) of sitagliptin succinate and its associated (values) respectively, as prepared by Example 5.

FIG. 10 depicts the Fourier-Transform Infra-Red (FTIR) spectrum of sitagliptin succinate prepared as per Example 5.

The X-ray powder diffractograms (XRPD) of the samples were determined by: Instrument: PANalytical; Mode: Expert PRO; Detector: Xcelerator; Scan Range: 3-40; Step size: 0.02; Range: 3-40 degree 2 theta; CuKα radiation at 45kV.

FTIR of the samples was determined by: Instrument: Perkin Elmer, SCAN: 16 scans, Resolution: 4.0 cm⁻¹, potassium bromide pellet method.

While the present invention has been described in terms of its specific embodiments, certain modifications and equivalents will be apparent to those skilled in the art and are intended to be included within the scope of the present invention.

### EXAMPLES

### Example 1

### Preparation of Amorphous Sitagliptin Maleate

Sitagliptin base (2 g, 0.0049 mole) was charged in methanol (36 mL) at 25° C. to 32° C. Maleic acid (0.57 g, 0.0049 mole) was charged at 25° C. to 32° C. to obtain a reaction mixture. The reaction mixture was stirred for 1 hour at 25° C. to 32° C. The reaction mixture was concentrated completely under vacuum at 45° C. to obtain a foamy solid. Hexanes (15 mL) were charged and stirred for 15 minutes at 25° C. to 32° C. The solid was filtered and washed with hexanes (10 mL). The solid was dried under vacuum at 40° C. for 16 hours to obtain the title compound.

Yield: 2.23 g (1.11 w/w, 86%)

### Example 2

### Preparation of Amorphous Sitagliptin Fumarate

Sitagliptin base (2 g, 0.0049 mole) was charged in methanol (36 mL) at 25° C. to 32° C. Fumaric acid (0.57 g, 0.0049 mole) was charged at 25° C. to 32° C. to obtain a reaction mixture. The reaction mixture was stirred for 1 hour at 25° C. to 32° C. The reaction mixture was concentrated completely under vacuum at 45° C. to obtain a foamy solid. Hexanes (15 mL) were charged and stirred for 15 minutes at 25° C. to 32° C. The solid was filtered and washed with hexanes (10 mL). The solid was dried under vacuum at 40° C. for 16 hours to obtain the title compound.

Yield: 2.45 g (1.22 w/w, 95%)

### Example 3

### Preparation of Amorphous Sitagliptin Benzenesulfonate

Sitagliptin base (2 g, 0.0049 mole) was charged in methanol (40 mL) at 25° C. to 32° C. Benzenesulfonic acid (0.77 g, 0.0049 mole) was charged at 25° C. to 32° C. to obtain a reaction mixture. The reaction mixture was stirred for 1 hour at 25° C. to 32° C. The reaction mixture was concentrated completely under vacuum at 45° C. to obtain a foamy solid. Hexanes (20 mL) were charged and stirred for 15 minutes at 25° C. to 32° C. The solid was filtered and washed with hexanes (10 mL). The solid was dried under vacuum at 40° C. for 16 hours to obtain the title compound.

Yield: 2.58 g (1.29 w/w, 93%)

### Example 4

### Preparation of Amorphous Sitagliptin Methanesulfonate

Sitagliptin base (2 g, 0.0049 mole) was charged in methanol (40 mL) at 25° C. to 32° C. Methanesulfonic acid (0.47 g, 0.0049 mole) was charged at 25° C. to 32° C. to obtain a reaction mixture. The reaction mixture was stirred for 1 hour at 25° C. to 32° C. The reaction mixture was concentrated completely under vacuum at 45° C. to obtain a foamy solid. Hexanes (15 mL) were charged and stirred for 15 minutes at 25° C. to 32° C. The solid was filtered and washed with hexanes (10 mL). The solid was dried under vacuum at 40° C. for 16 hours to obtain the title compound.

Yield: 1.96 g (0.98 w/w, 79%)

### Example 5

### Preparation of Amorphous Sitagliptin Succinate

Sitagliptin base (2 g, 0.0049 mole) was charged in methanol (40 mL) at 25° C. to 32° C. Succinic acid (0.57 g, 0.0049 mole) was charged at 25° C. to 32° C. to obtain a reaction mixture. The reaction mixture was stirred for 1 hour at 25° C. to 32° C. The reaction mixture was concentrated completely under vacuum at 45° C. to obtain a foamy solid. Hexanes (15 mL) were charged and stirred for 15 minutes at 25° C. to 32° C. The solid was filtered and washed with hexanes (10 mL). The solid was dried under vacuum at 40° C. for 16 hours to obtain the title compound.

Yield: 1.8 g (0.9 w/w, 70%)

1. Amorphous form of a sitagliptin compound of Formula I 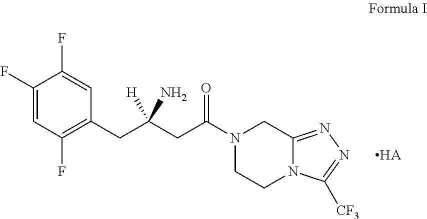
wherein HA is selected from the group consisting of maleic acid, fumaric acid, benzenesulfonic acid, methanesulfonic acid, and succinic acid.

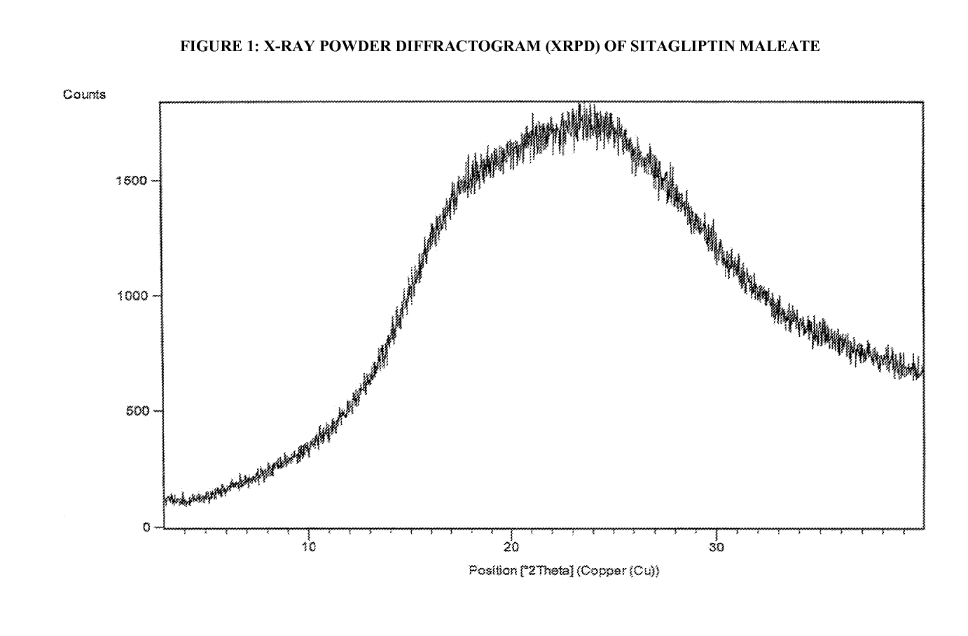

2. The amorphous form of sitagliptin maleate according to claim 1 characterized by an XRPD pattern substantially the same as depicted in FIG. 1.

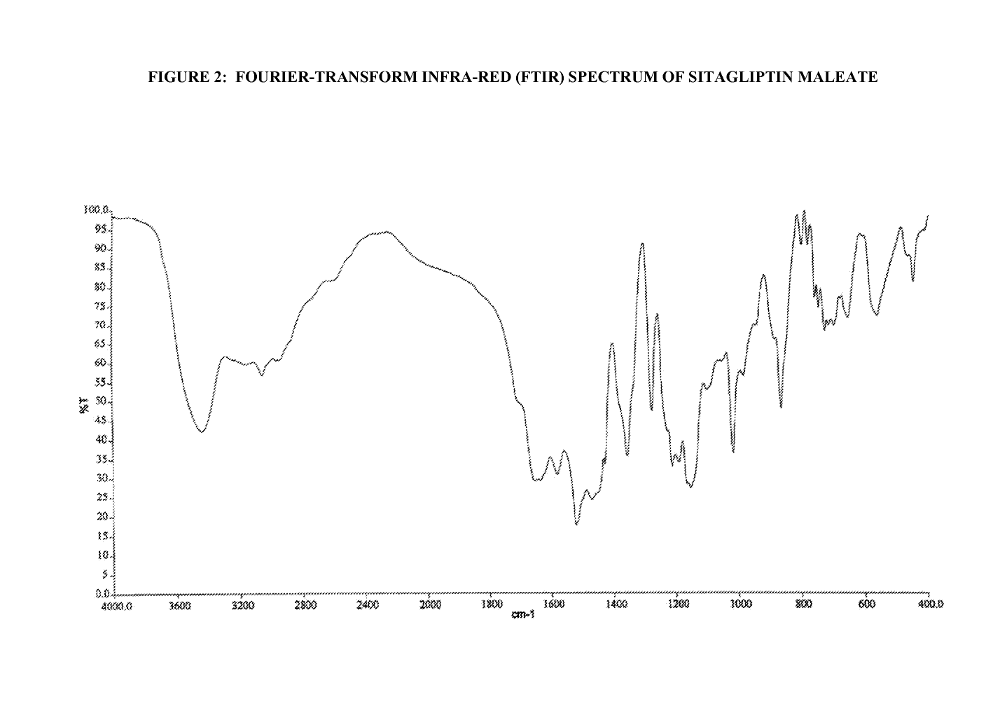

3. The amorphous form of sitagliptin maleate according to claim 1 characterized by FTIR as depicted in FIG. 2.

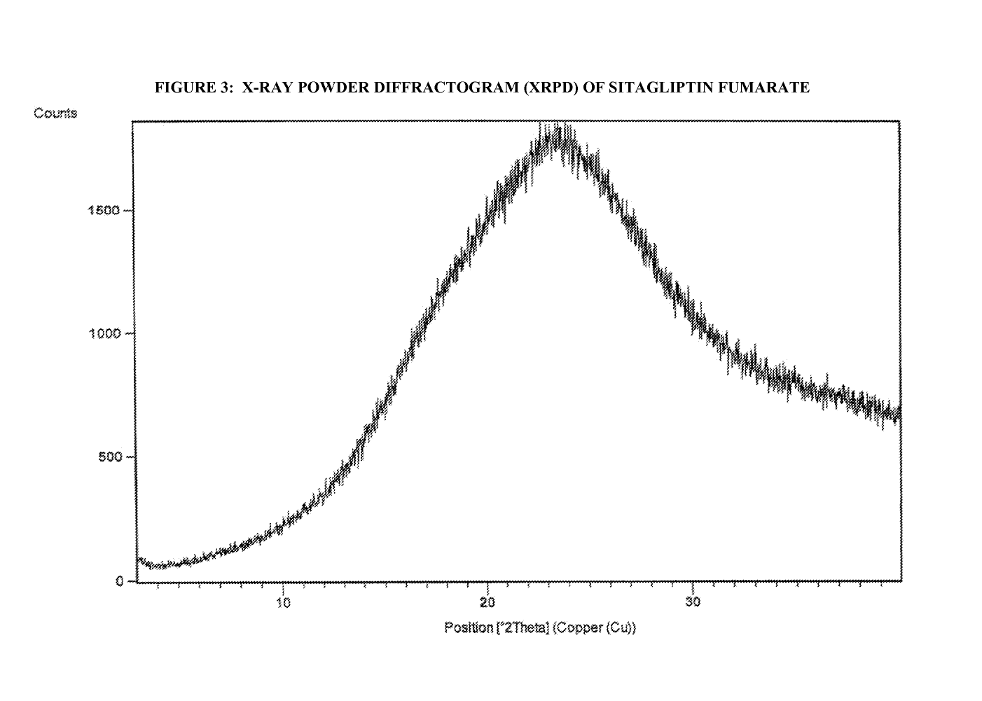

4. The amorphous form of sitagliptin fumarate according to claim 1 characterized by an XRPD pattern substantially the same as depicted in FIG. 3.

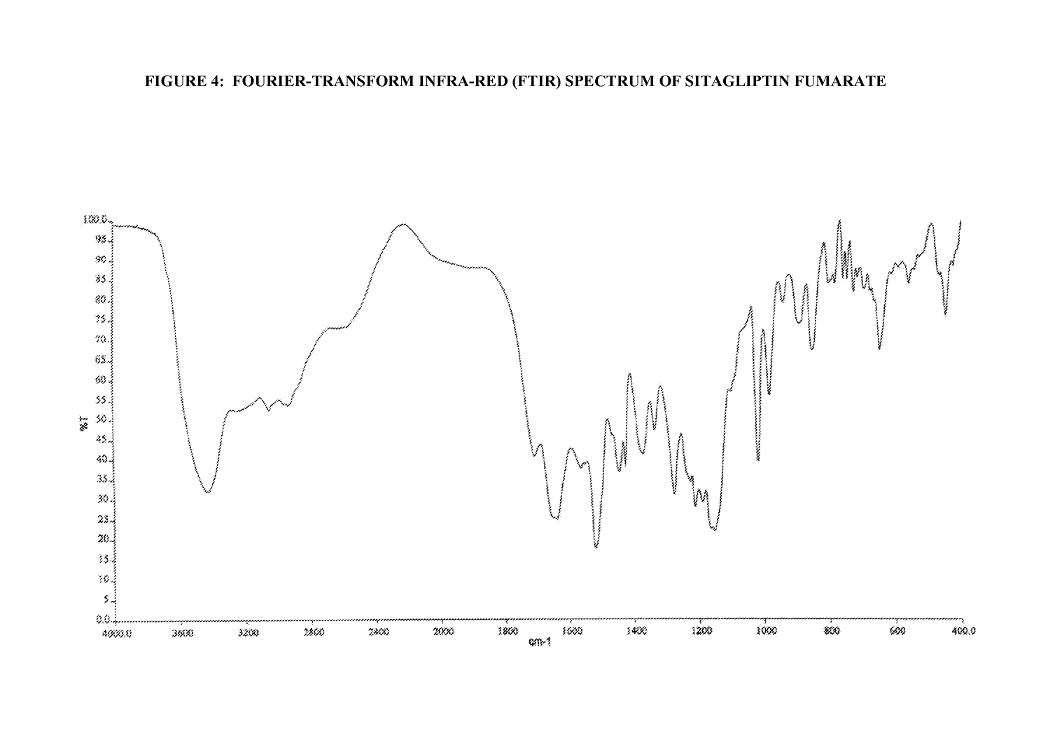

5. The amorphous form of sitagliptin fumarate according to claim 1 characterized by FTIR as depicted in FIG. 4.

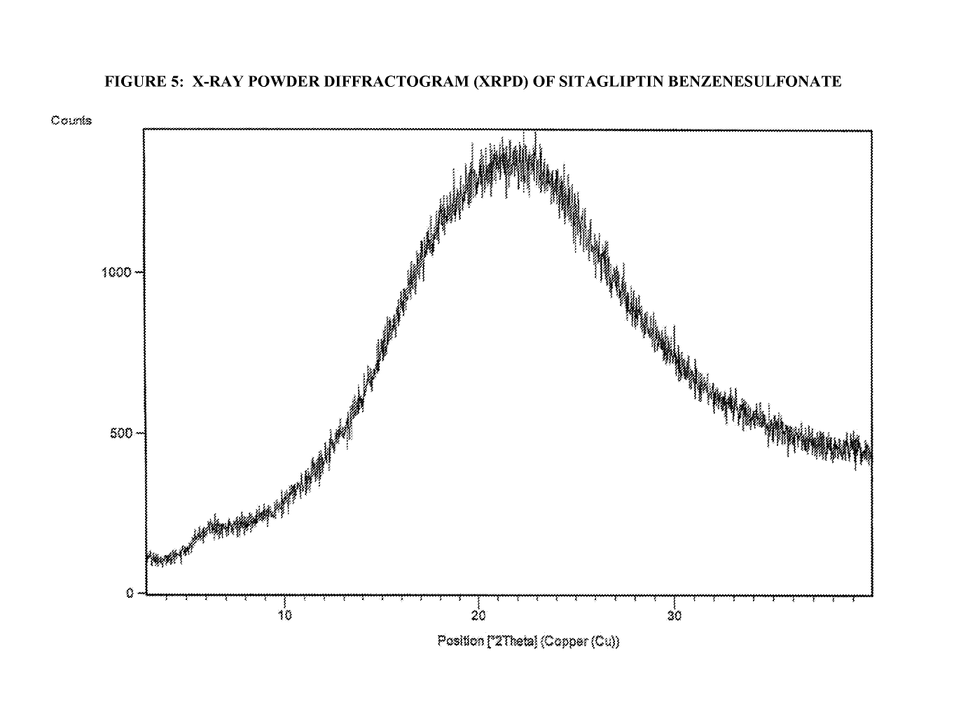

6. The amorphous form of sitagliptin benzenesulfonate according to claim 1 characterized by an XRPD pattern substantially the same as depicted in FIG. 5.

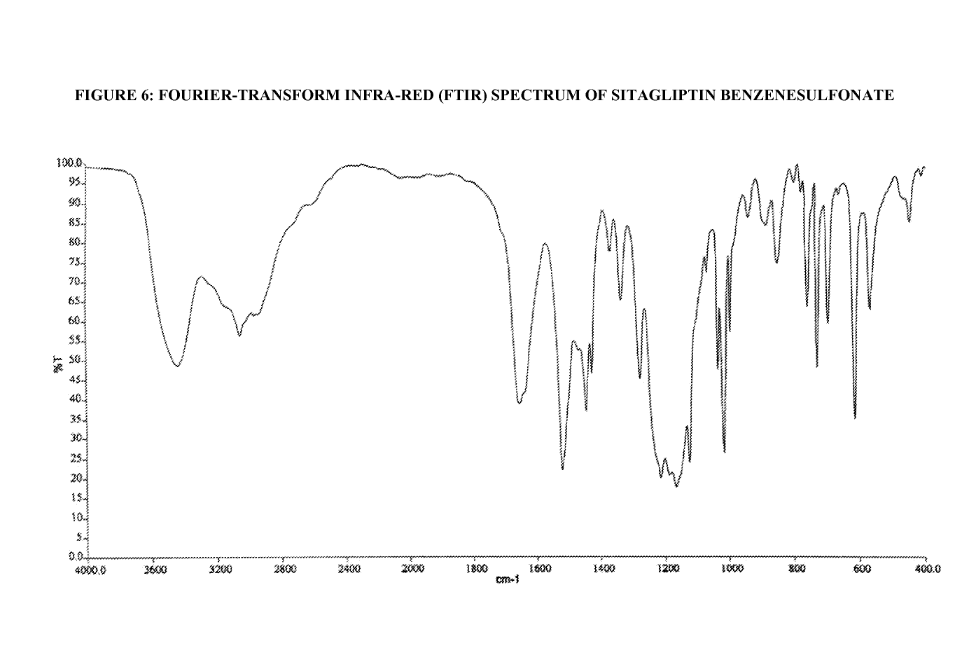

7. The amorphous form of sitagliptin benzenesulfonate according to claim 1 characterized by FTIR as depicted in FIG. 6.

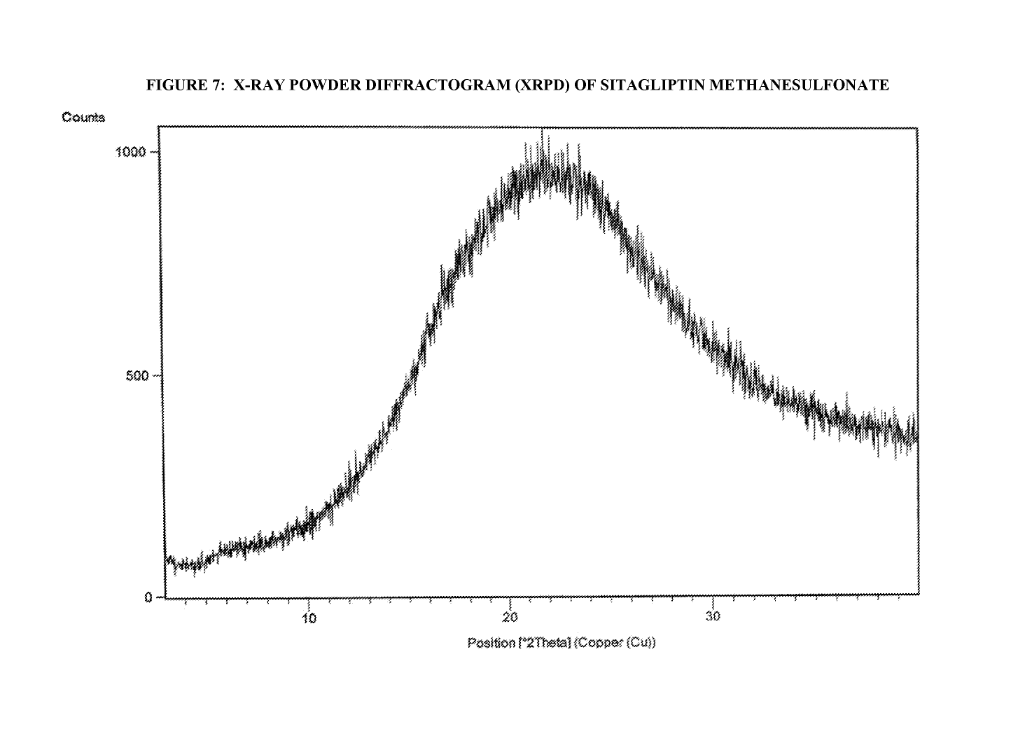

8. The amorphous form of sitagliptin methanesulfonate according to claim 1 characterized by an XRPD pattern substantially the same as depicted in FIG. 7.

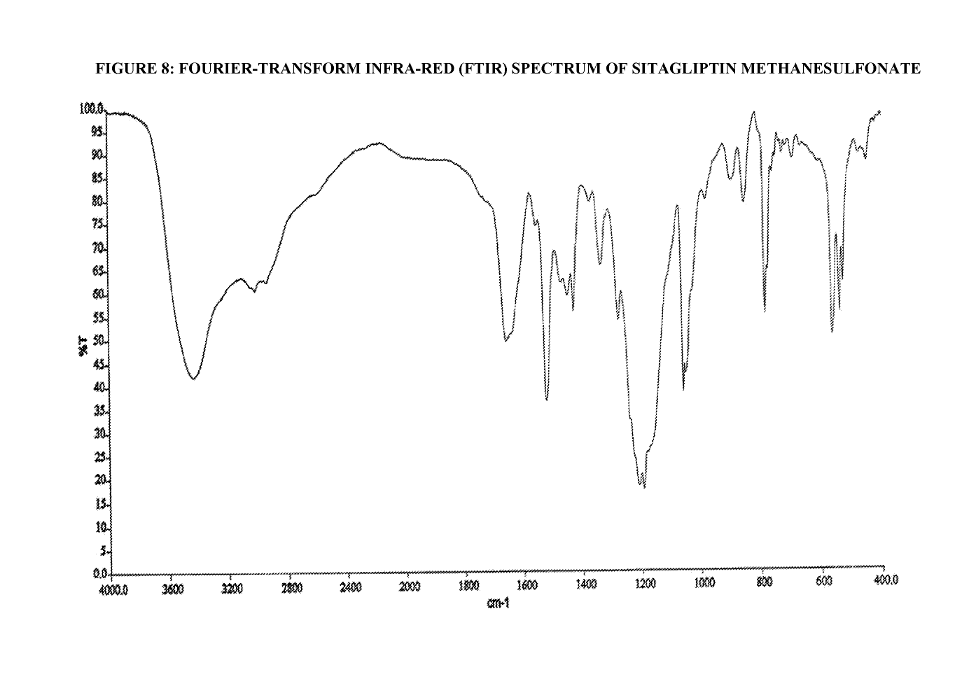

9. The amorphous form of sitagliptin methanesulfonate according to claim 1 characterized by FTIR as depicted in FIG. 8.

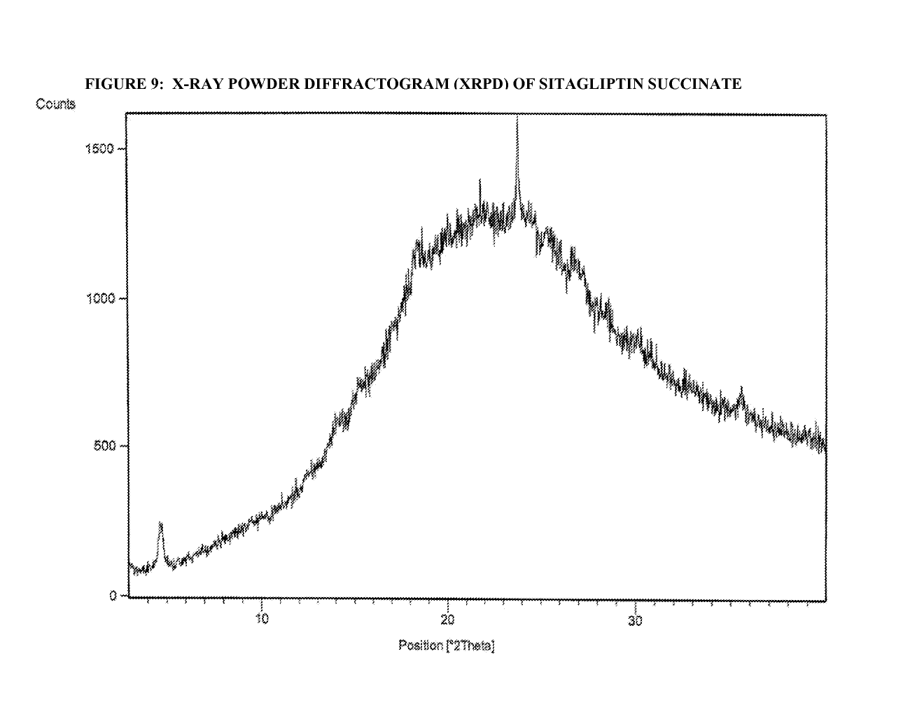

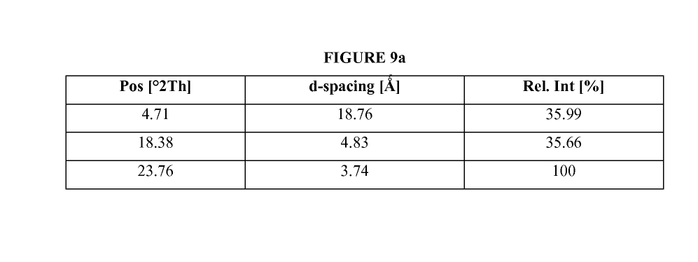

10. The amorphous form of sitagliptin succinate according to claim 1 characterized by an XRPD pattern substantially the same as depicted in FIG. 9.

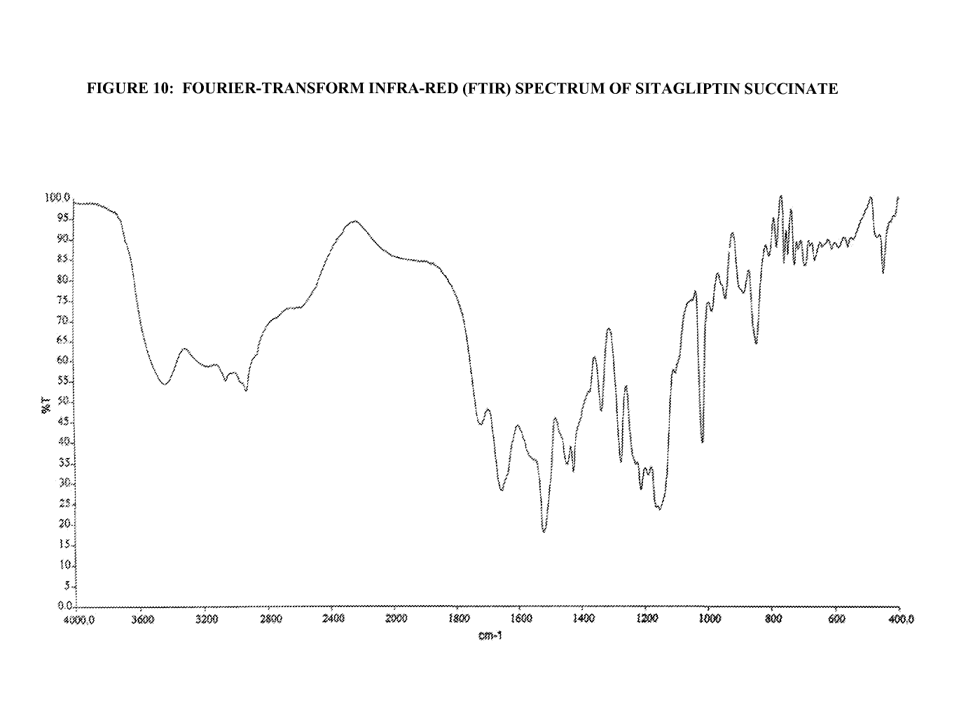

11. The amorphous form of sitagliptin succinate according to claim 1, characterized by FTIR as depicted in FIG. 10.

12. A process for the preparation of an amorphous form of the compound of Formula I 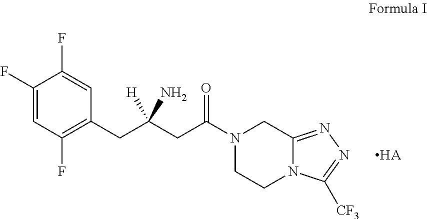
wherein HA is selected from the group consisting of maleic acid, fumaric acid, benzenesulfonic acid, methanesulfonic acid, and succinic acid, the process comprising:
a. treating sitagliptin with HA wherein HA is selected from the group consisting of maleic acid, fumaric acid, benzenesulfonic acid, methanesulfonic acid, and succinic acid; and
b. isolating an amorphous form of the compound of Formula I.

13. The process according to claim 12, wherein step a) of treating sitagliptin with HA includes adding, dissolving, slurrying, stirring, or a combination thereof

14. The process according to claim 12, wherein sitagliptin is treated with HA in a suitable solvent at a temperature of about 20° C. to about 80° C.

15. The process according to claim 14, wherein the solvent is selected from water, esters, alkanols, halogenated hydrocarbons, ketones, ethers, polar aprotic solvents, or mixtures thereof

16. The process according to claim 15, wherein the esters are selected from ethyl acetate, n-propyl acetate, isopropyl acetate, and n-butyl acetate.

17. The process according to claim 15, wherein the alkanols are selected from methanol, ethanol, n-propanol, isopropanol and butanol.

18. The process according to claim 15, wherein the halogenated hydrocarbons are selected from dichloromethane, chloroform, and 1,2-dichloroethane.

19. The process according to claim 15, wherein the ketones are selected from acetone and methyl ethyl ketone.

20. The process according to claim 15, wherein the ethers are selected from diethyl ether and tetrahydrofuran.

21. The process according to claim 15, wherein the polar aprotic solvent is selected from N,N-dimethylformamide, N,N-dimethylacetamide, dimethylsulphoxide, acetonitrile and N-methylpyrrolidone.

22. (canceled)

23. A pharmaceutical composition comprising the amorphous form of the sitagliptin according to claim 1, and a pharmaceutically acceptable carrier.

24. A method of treating or preventing type 2 diabetes mellitus comprising administering to a patient in need thereof a therapeutically effective amount of the amorphous form of the pharmaceutical composition according to claim 22.
